(19) United States
(12) Patent Application Publication
(10) Pub. No.: US 2009/0265971 A1
(43) Pub. Date: Oct. 29, 2009

(54) WRIST BAND IDENTIFICATION OF ACHIEVEMENT DEVICE AND METHOD

(76) Inventor: Arnold James Cook, Pittsburgh, PA (US)

Correspondence Address: ARNOLD J. COOK, 413 N. PASADENA DR., PITTSBURGH, PA, 15215, US

(21) Appl. No.: 12/110,303

(22) Filed: Apr. 26, 2008

(51) Int. Cl. A61B 5/117; A45F 5/00

(52) U.S. Cl. 40/633; 224/267

(57) ABSTRACT

A novel system and method is disclosed for an adjustable wristband and identification system for achievement. Disclosed is a system which includes attachable/removable features such as band, badges, and stripes which can be attached or removed from a wristband to identify accomplishment, rank, or position along with a method of production and use. Additionally the use of multiple colored bands and identifiers is disclosed to identify different branches or levels used in combination with identifiers which are removed or attached to show position within the level to create a complete system for identification of achievement.

### FIELD OF THE INVENTION

The present invention relates to the identification of achievement and the way that it is displayed for others to see. More specifically the present invention relates to the identification and display of achievement where identification can not easily be done with clothing or with items attached to clothing. The present invention also relates to helping to motivate people to achieve and respect the achievements of others.

### CROSS REFERENCE TO RELATED APPLICATIONS

Not applicable

### FEDERALLY SPONSORED RESEARCH OR DEVELOPMENT

Not applicable

### SEQUENCE OR COMPUTER PROGRAM

Not applicable

### BACKGROUND OF THE INVENTION

Achievement or mastery in any discipline is important. Throughout history identifiers have been created by man in a variety of disciplines to help others to see the accomplishments of the member, student, or teacher. Identifiers also help to demonstrate rank or the level of knowledge or authority a person has achieved. These identifiers can inspire as well as alert others with the information they provide.

In some groups such as the scouts, the military, or the police, identifiers are part of or added to the clothing that a person wears. Stripes, bars, badges, pins, and colors can all be parts of this identification system.

Karate schools use belts around a person's waist to identify a person's rank and level of skill. These belts are worn over top the clothing to make them easy to see. Karate schools have adopted a basic color system to identify accomplishments; beginning with white, then yellow, green, brown, and then ending with black. Many Karate schools that deal with younger students have adopted an even broader system: white, orange, green, purple, brown, and then black. In addition many schools use stripes on the belt to demonstrate rank or degree that person has accomplished at that belt level. Each stripe signifies something that the student has accomplished. Once, for example, a student has received 4 stripes on their belt, they are then ready to be tested for everything that belt level requires. If they pass, they then move to the next belt color and start the process again.

In other groups such as exercise classes no such ranking or identification system currently exists. There are many reasons for this. There are no standard uniforms or standardized requirements or ranking systems for exercise classes. There is a need for a ranking system that can be used for a wide range of activities to motivate and inspire, a system that can be easily displayed and understood.

Some sports like golf have a handy-cap system for ranking golfers. Tennis has a level system that ranks players by number 1, 2, 3, 3.5, 4, 4.5, and 5 so they can compete against people of similar skill level. Soccer and some other sports have A, B, C levels of players who compete in different leagues. These players are given their ranking by teachers, coaches, and/or organizations. These rankings are not currently displayed in any way by the individuals or teachers.

Professionals in most areas now have one or more certifications that the person must have in order to do certain jobs, from plumbers to doctors they all have certifications and no way of simply displaying them. The present invention provides a way for these certifications as well as the area of the certification to be displayed and easily modified.

### SUMMARY OF THE INVENTION

The object of the present invention is to provide a system for the display of achievement in a discipline and to do so without adding items to clothing such as badges, pins, or stripes or impinging on the person's ability to move.

The present invention utilizes a series of wrist bands and band add-ons to create a wearable ranking system similar to the waist belt system used by karate schools. The present invention makes it possible to easily identify the discipline as well as the level the person has achieved at that particular discipline. The system of the present invention provides for different wrist band levels as well as different levels or achievements within each wrist band level.

The present invention relates to any discipline where displaying the level of competency is desired without modifying the clothing or requiring special clothing or additions to the clothing. The present invention can also be used in the training of students and to help motivate them.

The present invention also relates to training the body as well as the mind. For example in the field of exercise and in sports the present invention provides a way for teachers, trainers, and coaches to organize their instruction into measurable levels with identifiable and testable steps within each level. The present invention provides a way for the member, student, and teacher to demonstrate their level in that discipline so that anyone can easily see what level that person has achieved.

The present invention discloses an wrist band system as well as a method to use and a method to produce the wrist band system. The present invention provided an wrist band which can be worn to demonstrate the level of accomplishment in a discipline and which can be added to demonstrate accomplishments within that level. Also a multiple color/multiple level system is disclosed.

The present invention discloses the use or different colors and/or shapes and symbols on the wrist bands for each level. In addition the present invention discloses the use of identifiers, which disclose the discipline itself, and the position within the level of the discipline or certification(s) within a discipline. In one embodiment, the beginning student starts with level wrist band one which signifies that the person has not yet been evaluated. After some evaluation the teacher can elect to have the student tested and awarded the appropriate wrist band.

The present invention incorporates identifiers, which can be added to or removed from the wrist bands. More than one identifier can be added to the wrist band. Also more than one discipline can be displayed on one wrist band. Also multiple wrist bands may be worn.

The present invention may be used to display military rank by incorporating stripes, bars, and stars similar to those currently used on military clothing. Colors can be used for example to identify the branch of the military, as is currently done with clothing. In addition symbols may be incorporated on the wrist band to designate for example the group within the branch.

The present invention may be used for scouts or other groups in the same manner and incorporate the identifiers normally worn on clothing. The present invention may be used by karate schools as well to display the ranks and accomplishments in the same way as the waist belts currently do.

In addition the present invention can be used to demonstrate accomplishments for any teaching or discipline. Also multiple disciplines or sub areas and levels can be displayed simultaneously on the same wrist band. Also an identifier on the wrist band can identify letters in a school sport for a school such as awards in science, etc.

The present invention also disclosed the use of a color wrist band system starting with white and moving through the colors to black. Additional details, features, and advantages of the present invention will be set forth in the description which follows, and will be made obvious from the description and the use of the present invention. Advantages and features of the invention may be obtained and realized by parts and combinations particularly pointed out in the appended claims. These and other features of the present invention will become more fully apparent from the following description and appended claims, or may be learned by the practice of the present invention as set forth herein.

In addition a method for using the present invention will be set forth in the description which follows. The advantages and features of the method may be realized through the description and appended claims and may also be obtained by the practice of the method for the present invention as set forth herein.

### BRIEF DESCRIPTION OF DRAWINGS

In order to describe the manner in which the above-recited and other advantages, method, and features of the present invention can be obtained a more particular description of the invention briefly described above will be rendered by reference to specific embodiments there of which are illustrated in the appended drawings. Understanding that these drawings depict only typical embodiments of the invention and are not therefore to be considered to be limiting in scope, the invention will be described and explained with additional specificity and detail through the use of the accompanying drawings in which:

FIG. 1 illustrates an exemplary embodiment of the achievement identification device in accordance with the present invention on the arm of a user.

FIG. 2 illustrates an exemplary embodiment where the achievement identification device is removed from on the arm of the user, showing how the stripes are attached.

FIG. 3 illustrates an exemplary embodiment of the side view of the achievement identification device laid out flat.

FIG. 4 illustrates an exemplary embodiment providing a detailed view of the adjusting snaps for adjusting the size to fit different wrist sizes.

FIG. 5 illustrates an exemplary embodiment providing a detailed view of alternative snap designs.

FIG. 6 illustrates an exemplary embodiment showing a military armband with stripes to show rank and a center section to show the military branch.

FIG. 7 illustrates an exemplary embodiment showing that the center badge and ranking stripes can be added or removed.

FIG. 8 illustrates an exemplary embodiment showing an alternative achievement arm band design with snap on center badge and slide on stripes.

FIG. 9 illustrates an exemplary embodiment showing different colors or patterns may be used to identify rank.

FIG. 10 illustrates an exemplary embodiment showing different symbols on the ranking stripes.

FIG. 11 illustrates an exemplary embodiment of the present invention detailing an armband laid out flat with a center badge and removable stripes.

### DETAILED DESCRIPTION

The present invention relates to an identification system used to show the level of rank or achievement in a particular discipline and more particularly a way to provide identification of such information without special clothing or additions on to other clothing. This system and the disclosed device use an wrist band with attachable identifiers that can for example display the ranking, special awards, or level of skill or authority in one or multiple disciplines.

The following description of the present invention utilizes a series of diagrams that illustrate the structure of a number of exemplary embodiments for implementing the present invention. Using the diagrams in this manner to present the invention is for illustration purposes only and should not be construed as limiting the scope of the present invention.

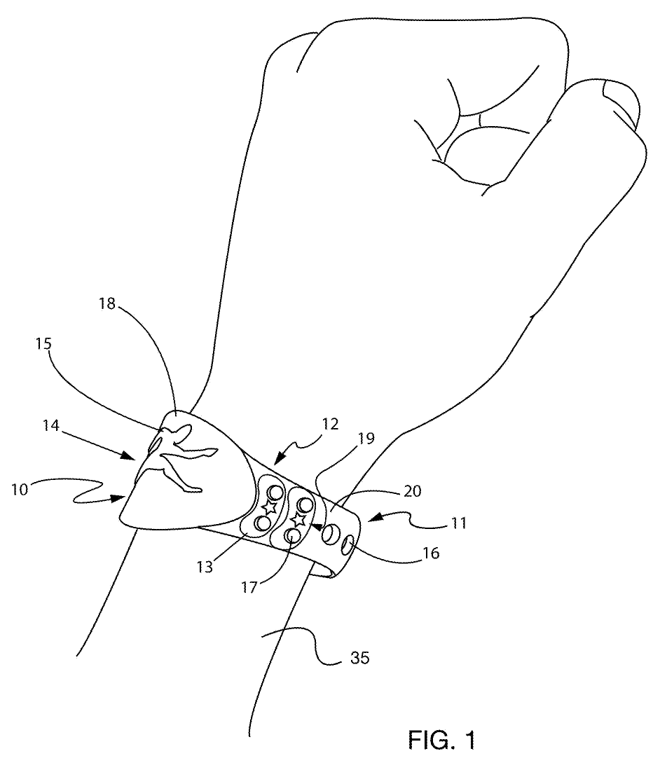

In FIG. 1 is an exemplary embodiment of the present invention in which an achievement identification system 10 fits around the wrist for a user 35. The achievement identification system 10 has an adjustment system 11 to fit different size wrists and an identifier system 12 with attachable and removable identifiers 13 in addition to the achievement identification system 10 may include a descriptor system 14. In FIG. 1 the achievement identification system 10, adjustment system 11 comprises a preferred embodiment with adjustment holes 16 used to select the size of the wrist band 20, similar to the sizing in a watch band. Many different mechanisms for adjusting the size may be utilized including snaps, hook and loop fasteners, a buckle with a tongue that fits into the adjustment holes 16, or a clamp which clamps the wrist band 20 together. In addition a stretchable polymer, fabric, metal, spring, or other mechanism may be used to allow the wrist band 20 to stretch to accommodate different size wrists without an opening.

In this embodiment the identifiers 13 snap onto posts 17, the identifiers 13 and the posts 17 are made of an elastic polymer such as silicon rubber, PVC (Polyvinyl Chloride) or other thermoplastic or thermoset polymer. In the preferred embodiment, PVC is used to form the wrist band 20 and the identifiers 13. PVC wrist band 20 can have multiple different colors molded in different colors may be easily molded in by filling different areas of the mold with different colors and then curing the whole system together. FIG. 1 shows a descriptor system 14 where there is molded in a logo 15 molded in one color and a background 18 out of a different color.

The descriptor system 14 can be used to describe the field, sport, branch, department and so forth that the identifiers 13 apply to. The identifiers 13 can include their own identifier logo 19. The identifiers 13 when attached demonstrate a rank, accomplishment, certification, position or other trait of meaning.

For example the descriptor system 14 may identify the branch of military service such as the Navy while the identifiers 13 which attach may identify the rank or accomplishment of the soldier.

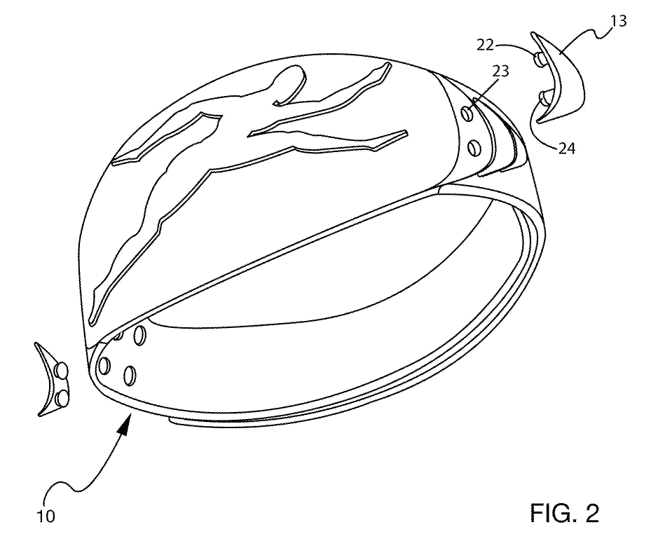

FIG. 2 is an exemplary embodiment of the present invention wherein the achievement identification system 10 includes identifiers 13 winch connect on to the wrist band 20. In this preferred embodiment the identifiers 13 have posts 22 which snap into post holes 23 or the reverse the identifiers may have hole which snap on to posts. The post 22 have a stop ring 24 which is bigger in diameter than the post hole 23. The post hole 23 expands to allow the post 22 to enter the post hole 23 and once installed the post ring 24 keeps the post 22 from coming out. The dimensions of the posts and holes as well as the durometer of the polymer can all be modified to adjust the force required to install or remove the identifiers 13. Hook and loop fasteners such as those sold under the trade name of Velcro as well as other attachments methods such as adhesive, mechanical snaps, or combinations of attachments methods may also be used to attach the identifiers 13.

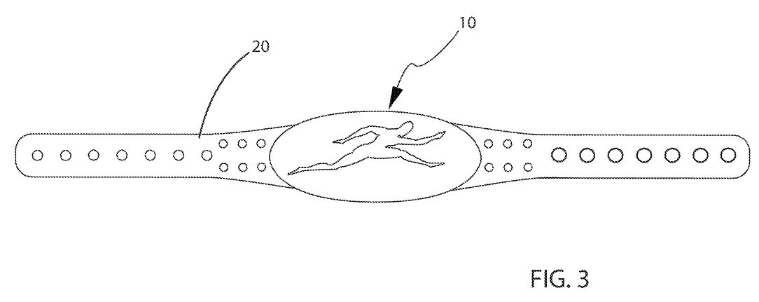

FIG. 3 is an exemplary embodiment of the present invention achievement identification system 10 with the wrist band 20 laid out flat to show the different details. The wrist band 20 can be molded flat or die cut out of a sheet of polymer. Preferably the wristband 20 is cast out of PVC (poly vinyl chloride) in a flat position by pouring (filling) different areas with the selected colors and durometers.

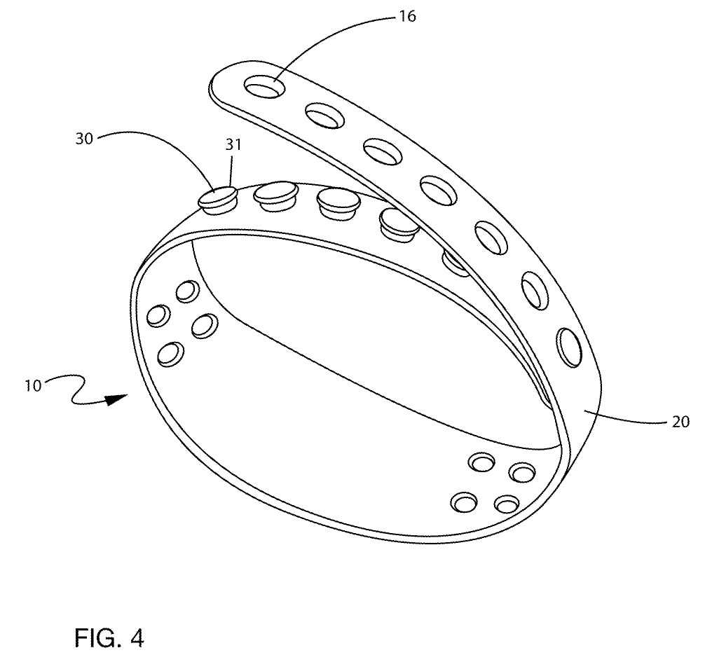

FIG. 4 is an exemplary embodiment of the present invention with the wrist band 20 removed from the mold showing how the adjustment posts 30 fit into the adjustment holes 16. The adjustment holes 16 stretch over the adjustment posts 30 such that the post rings hold the adjustment post 30 in the adjustment holes 16.

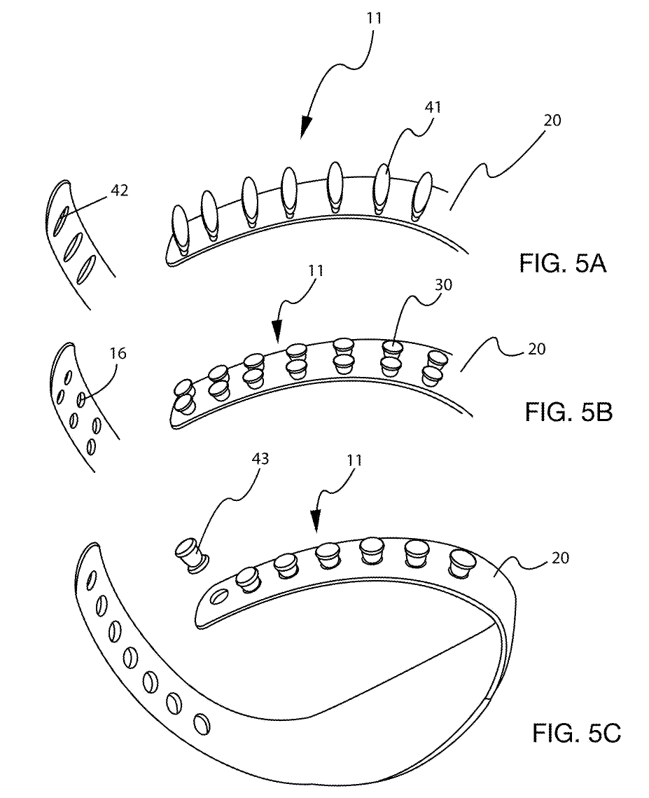

FIGS. 5A, B, and C are exemplary embodiments of the attachment system 11 of the present invention showing different post and hole designs.

FIG. 5A illustrates elongated post 41 and elongated holes 42 which snap together to attach the ends of the wrist band 20.

FIG. 5B illustrates a dual set of adjustment posts 30 and adjustment holes 16 for added strength. Preferably the posts are between 1/16″ and ¼″ in diameter, however posts larger than ¼″ and smaller than 1/16″ in diameter may be used.

FIG. 5C illustrates separate connector posts 43 that can be used to connect the ends of the wrist band 20 together. The connector posts 43 may be made of the same material as the wrist band 20 or a different material such as a harder polymer or metal.

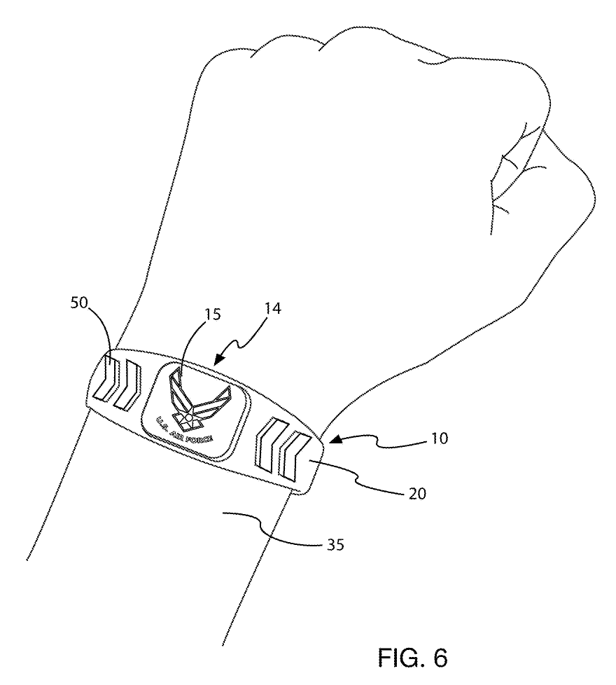

FIG. 6 is an exemplary embodiment of the present invention showing the achievement identification device 10 with the wrist band 20 including the logo 15 of the army with stripes 50 to show rank of the user 35. The color of the wrist band 20 can also be used to identify rank or a subgroup of the descriptor system 14 group such as a branch or division of the army.

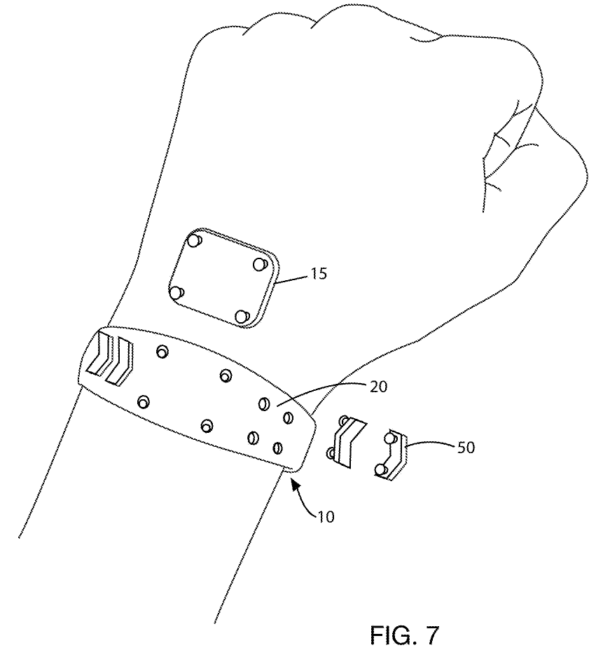

FIG. 7 is an exemplary embodiment of the present invention where the logo 15 and the stripes 50 are not attached, such that a different logo 15 can be attached and stripes 50 or other badges can be added or removed.

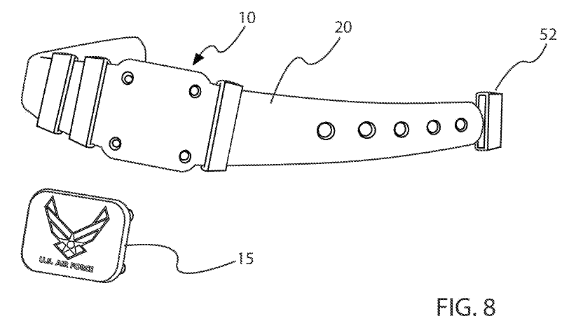

FIG. 8 is an exemplary embodiment of the present invention where instead of snap on stripes as shown in FIG. 7, slide on bands 52 can be slid onto the wrist band 20 to identify achievements.

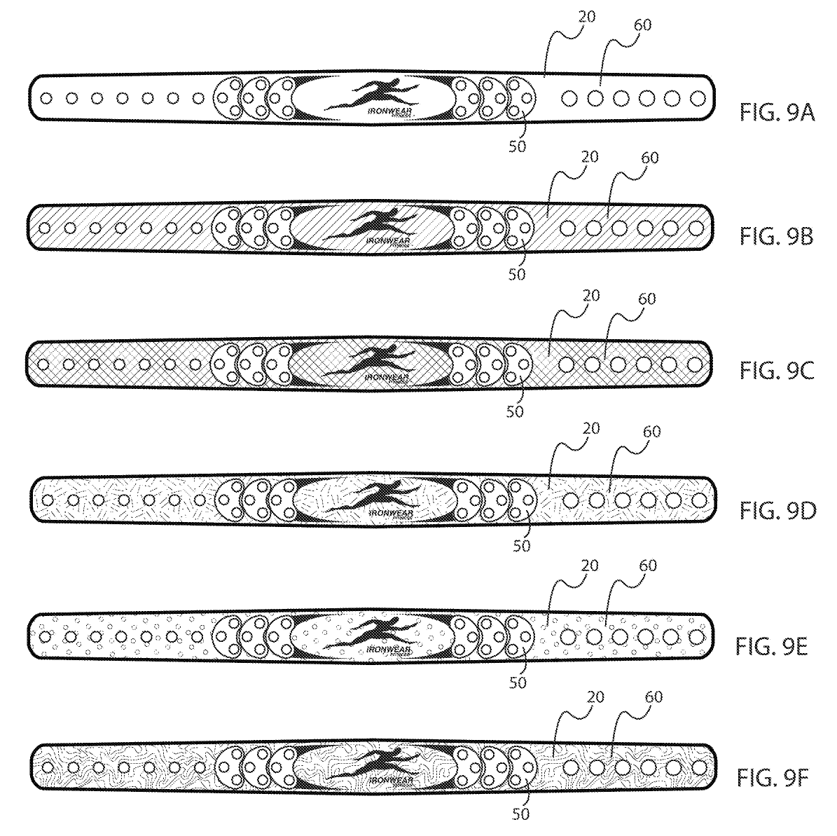

FIGS. 9A, B, C, D, E and F identify that different colors or patterns can be used along with stripes for example to show rank. FIG. 9A represents light color 60 such as white, clear, or yellow wrist band 20 which can identify a beginning or unranked person in the discipline.

FIG. 9B is a red or orange color 61 to identify a person that has begun their training and has passes some basic tests.

FIG. 9C represents a green color 62 or middle pattern that a person has reached an intermediate level.

FIG. 9D represents a purple color 63 demonstrating a person is between intermediate and advanced in ranking.

FIG. 9E represents brown or gray color 64 that a person has achieved an advanced level and that they are working towards a master level.

FIG. 9F represents a black color 65 and the most advanced level. Within each of these levels strips 50 can be used to show steps or tests the person has passed already on their way to the next wrist band 20 color.

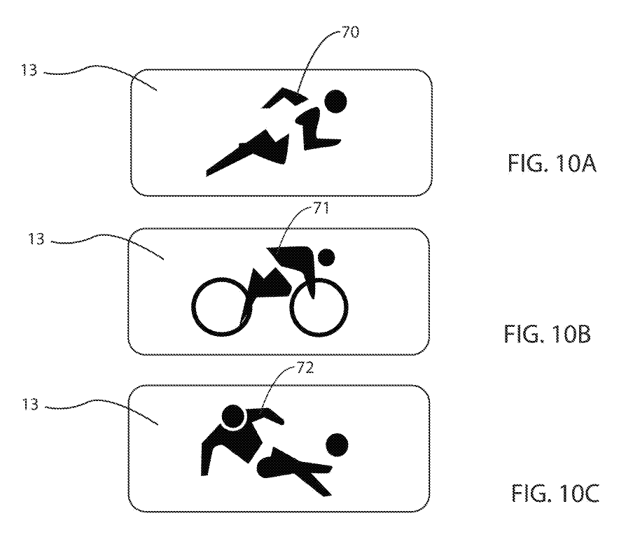

FIGS. 10 A, B, and C represents different identifiers 13 or different symbols that can be used to identify for example merit badges that a scout might earn by meeting different merit badge requirements. Logos can be used to identify different sports that the wrist band 20 could show the rank in, for example FIG. 10A for running 70, FIG. 10B for cycling 71, and FIG. 10C for soccer 72.

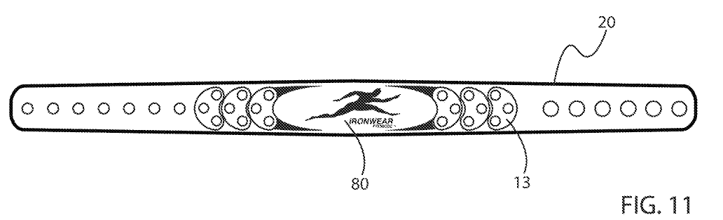

FIG. 11 is an exemplary embodiment of the wrist band 20 of the present invention laid out flat showing the Ironwear Fitness logo 80 and snap on identifiers 13 to demonstrate accomplishment in the use and training with Ironwear Fitness equipment.

The present invention is a device for identification of achievement comprising; a wrist band worn around the wrist of a user; and attachable and removable achievement identifiers that can be attached to or removed from the wrist band. Also included is wrist band as described wherein the band is an elastic polymer, a wrist band wherein the identifiers show the rank or accomplishments of the user, a wrist band wherein colors or patterns are used to identify rank. In addition the can attach by snapping onto the wrist band with posts.

The present invention includes a wrist band wherein the wrist band has the colors to signify the level or the user in a disciple including the following color system wherein white, clear, or yellow represent that the user is untested or just starting, orange or red represents beginning, green represents intermediate, purple represents advanced intermediate, brown or gray represents advanced, and black represents expert.

The present invention includes identifiers which attach to the wrist band wherein the identifier is one or more stripes or other markings which demonstrate accomplishments within a discipline. The identifiers can identify the area of the accomplishment group, activity, sport, or discipline.

The present invention includes a wrist band for the identification of accomplishment within a group or discipline; and an identification system on the wrist band which identifies the group or discipline; and a ranking system which is attached or removed from the band to identify a person's accomplishments or rank within the group or discipline.

The present invention includes a wrist band which is worn around a user's wrist to identify accomplishments of the wearer and an identification system which can be changed to demonstrate changes in the user's area of accomplishment.

The present invention includes a wrist band that can be made of fabric, metal, stone or plastic preferably the wristband the wristband is comprised of a flexible polymer. The polymer can be thermoformed or thermoset, preferably the polymer is polyvinyl chloride, or silicon rubber.

The present invention includes the method of identifying a person's accomplishment including wearing a wrist band which: has a marking system that demonstrates the group; and attaching or removing identifiers to demonstrate the rank or accomplishments within the group or discipline. And a method wherein the person attaches a stripe or other identifier each time they achieve a new ranking. The present invention also includes a method wherein the user switches the color of the wristband to demonstrate a higher rank after they have earned all requirements from the prior wrist band color.

The present invention includes a wrist band to identify the position in the discipline of karate, the military, the position of a person in a sport, a profession, or a club.

The present invention includes a flat wrist band produced by forming or casting of a polymer of different colors into a mold; and creating post at one end of the band, and holes at the other end such that the post fits into the holes; and attaching the band around a person's wrist to identify rank or position.

Although the invention has been described in detail in the foregoing embodiment for the purpose of illustration, it is to be understood that such detail is solely for that purpose and that variations can be made therein by those skilled in the art without departing from the spirit and scope of the invention except as it may be described by the following claims.

What is claimed is:

1. A device for identification of achievement comprising;
A wrist band worn around the wrist of a user; and
Attachable and removable achievement identifiers that can be attached to or removed from the wristband to show the achievements of the user.

2. A wrist band as described in claim 1 wherein the band is an elastic polymer.

3. Identifiers as described in claim 1 wherein the identifiers show the rank or accomplishments of the user.

4. A wrist band as described in claim 1 wherein colors or patterns are used to identify rank.

5. An identifier as described in claim 1 wherein the identifier snaps onto the wrist band with posts.

6. Wrist band as described in claim 4 wherein the wrist band has the colors white, clear, or yellow representing untested or just starting, orange or red representing beginning, green representing intermediate, purple representing advanced intermediate, brown or gray representing advanced, and black representing expert.

7. An identifier as described in claim 1 wherein the identifier is one or more stripes which demonstrate accomplishments within a discipline.

8. Identifiers as described in claim 1 which identify the area of the accomplishment group, activity, sport, or discipline.

9. A wrist band for the identification of accomplishment within a group or discipline;
An identification system on the wrist band which identifies the group or discipline; and
A ranking system which is attached or removed from the wrist band to identify a person's accomplishments or rank within the group or discipline.

10. A wrist band as described in claim 2 wherein the polymer is polyvinyl chloride.

11. A wrist band as described in claim 2 wherein the polymer is silicon rubber.

12. A method of identifying a user's accomplishment including wearing a wrist band:
A marking system on the wrist band that demonstrates the area of disciple; and
Attaching or removing identifiers to the wrist band to demonstrate the rank or of the user within the group or discipline.

13. A method as described in claim 12 wherein the user attaches a stripe or other identifier each time the user achieves a new ranking.

14. A method as described in claim 12 wherein the user switches the color of the wristband to demonstrate a higher rank after the user has earned all the ranking identifiers from one wrist band color.
